(19) United States
(12) Plant Patent Application Publication
(10) Pub. No.: US 2017/0295680 P1
(43) Pub. Date: Oct. 12, 2017

(54) Kiwi plant named 'HFY01'

(50) ;

(71) Applicant: Sichuan Hua Sheng Agriculture Co., Ltd., Chengdu (CN)

(72) Inventor: Li Ming Zhang, Chengdu (CN)

(73) Assignee: Sichuan Hua Sheng Agriculture Co., Ltd., Chengdu (CN)

(21) Appl. No.: 15/530,986

(22) Filed: Apr. 4, 2017

(30) Foreign Application Priority Data
Apr. 6, 2016 (QZ) 2016/0898

(51) Int. Cl. A01H 5/08

(52) U.S. Cl. CPC A01H 5/08
USPC PLT/156

(57) ABSTRACT

A new and distinct Actinidia plant named ‘HFY01’, particularly characterized by its strong growth habit, end-April time to bloom and early-December time to harvest, fruit size and shape, and PSA resistance.

Latin name of the genus and species of the plant claimed: Actinidia chinensis pl.

Variety denomination: ‘HFY01’.

### BACKGROUND OF THE INVENTION

The present invention relates to a new and distinct cultivar of Actinidia plant, botanically known as Actinidia chinensis pl of the Aclinidiacea family, and hereinafter referred to by the variety denomination ‘HFY01’.

The new Actinidia cultivar is a product of a controlled breeding and pollination program conducted by the inventor, in Xia Yuan Village, Shifang county, Sichuan Province, China. The objective of the breeding and pollination program was to develop a new Actinidia cultivar with a dark yellow fruit flesh color.

The new Actinidia cultivar originated from a open pollination of JXFX-CK-04136 (seed bearing parent only) made by the inventor(s) in Xia Yuan Village, Shifang County, Sichuan province, China. The female or seed parent is the Actinidia chinensis pl. cultivar designated JXFX-CK-04136 (seeds collected from wild-grown trees in Fengxin county, Jiangxi province, China, in 2004.). The variety was developed through open pollination and the male or pollen parent is not known.

Seeds were sown in February 2005 and 300 seedlings were then planted out in a field in Shifang County, Sichuan Province, China in February 2005. The seedlings first fruited in in April 2008. A total of 1 female seedlings were propagated by graft in October 2009 The new Actinidia cultivar was selected by the inventor(s) in a controlled environment in 2010 in Shifang County, Sichuan Province, China.

Asexual reproduction of the new Actinidia cultivar by grafting on to rootstocks of Actinidia deliciosa pl was first performed in April 2012 in a kiwifruit research station in Shifang county Sichuan province, China, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘HFY01’ which in combination distinguish this Actinidia as a new and distinct cultivar:
1. Very strong growth habit. In soil of PH 5-6.5 and annual average temperature of 16-18° C., the claimed variety will fruit in the second year after grafting and can produce up to 20-30 t/ha of fruit by the fourth year of fruiting.
2. Flowering time and blooming period. Flowering time is in about end-April in Sichuan Province, China and the blooming period is about 5 to 8 days.
3. Harvesting time. The harvesting time is in early-December in Sichuan Province, China.
4. Fruit Shape and Size. The general fruit shape is long-trapezium with a weakly depressed stylar end. The average fruit weight is 85 g and maximum fruit weight is 120 g;
5. Pest resistance. The claimed variety shows strong resistance to PSA.

In comparison to the parental variety, JXFX-CK-04136’ (unpatented), ‘HFY01’ differs primarily in the traits listed in Table 1.

TABLE 1

| | New Cultivar | Female Parent | Male Parent |
|---|---|---|---|
| Trait | ‘HFY01’ | ‘JXFX-CK-04136’ | Open pollen |
| flavor | sweet | acid | No fruit |
| Average fruit | 85 g | 60 g | No fruit |
| weight | | | |
| Fruit shape | long-trapezium | elliptic | No fruit |
| Color of flesh | Dark yellow | Green yellow | No fruit |
| shape of core | triangle | circular | No fruit |
| General shape | Slightly depressed | Deeply depressed | No fruit |
| of stylar end | | | |
| Plant resistance | More resistance | Less resistance | |

Of the many commercial varieties known to the present inventor(s), the most similar in comparison to the new Actinidia cultivar ‘HFY01’ is the variety ‘Jing Feng’ (unpatented), in the following characteristics

TABLE 2

| | New Cultivar | Comparison Cultivar |
|---|---|---|
| Characteristic | ‘HFY01’ | ‘Jing Feng’ |
| Average fruit | 85 g | 90 g |
| weight | | |
| Fruit shape | long-trapezium | elliptic. |
| General shape of | Slightly depressed | Weekly blunt |
| stylar end | | protruding |
| Color of inner | dark Yellow | medium yellow |
| pericarp (locules) | | |
| general shape of | triangle | transverse elliptic |
| core in cross | | |
| section | | |
| young leaf | strong. | week |
| petioles | | |
| anthocyanin | | |
| coloration | | |
| Plant resistance | More resistance | Less resistance |
| Sugar content (at | 17 to 18 Brix | 13 to 14 Brix |
| picking time): | fruit sweetness is | Fruit sweetness is very |
| | very hight | low, |
| Acidity/Starch | 1 to 1.4 g/lt | 1.5-2.0 g/lt |
| (at picking time) | Fruit acidity is low | Fruit acidity is high |

### BRIEF DESCRIPTION OF THE DRAWINGS

The accompanying photographs illustrate the overall appearance of the new Actinidia cultivar ‘HFY01’ showing the colors as true as is reasonably possible with colored reproductions of this type. Colors in the photographs may differ slightly from the color values cited in the detailed botanical description, which accurately describe the color of ‘HFY01’. Plant parts shown in the photographs are from approximately 4 year old plants.

FIG. 1. Shows typical whole fruit of the comparison variety ‘JING FENG’ (left) compared to typical whole fruit of the claimed variety ‘HFY01’ (right).

FIG. 2. Shows a cross-sectional view of typical whole fruit of the comparison variety ‘JING FENG’ (left) compared to typical whole fruit of the claimed variety ‘HFY01’ (right).

FIG. 3A. Shows the upper side of a typical leaf of the comparison variety ‘JING FENG’ (left) compared to a typical leaf of the claimed variety ‘HFY01’ (right).

FIG. 3B. Shows the under side of a typical leaf of the comparison variety ‘JING FENG’ (left) compared to a typical leaf of the claimed variety ‘HFY01’ (right).

FIG. 4A. Shows the upper side of typical flowers of the comparison variety ‘JING FENG’ (left) compared to typical flowers of the claimed variety ‘HFY01’ (right).

FIG. 4B. Shows the under side of typical flowers of the comparison variety ‘JING FENG’ (left) compared to typical flowers of the claimed variety ‘HFY01’ (right).

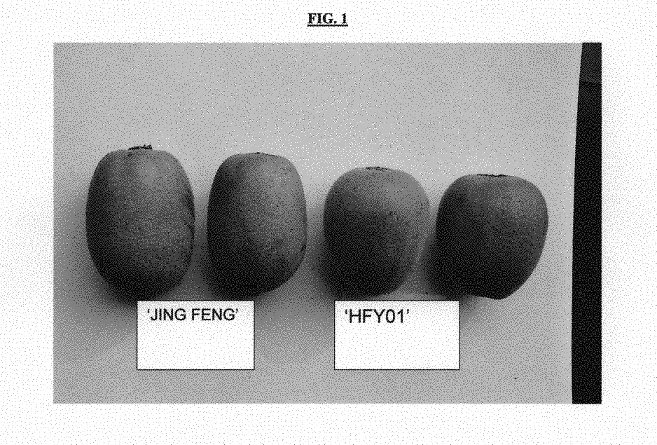

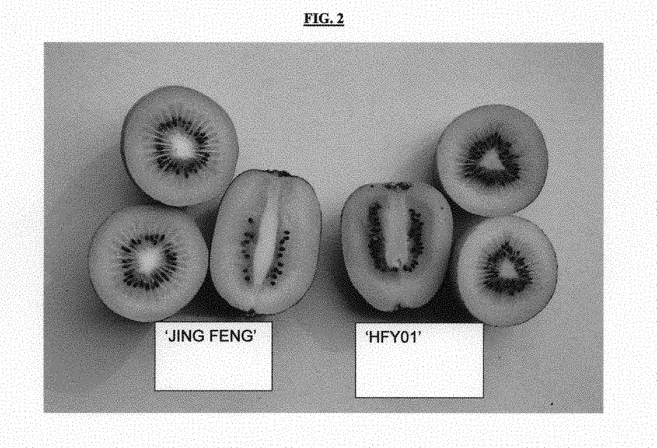

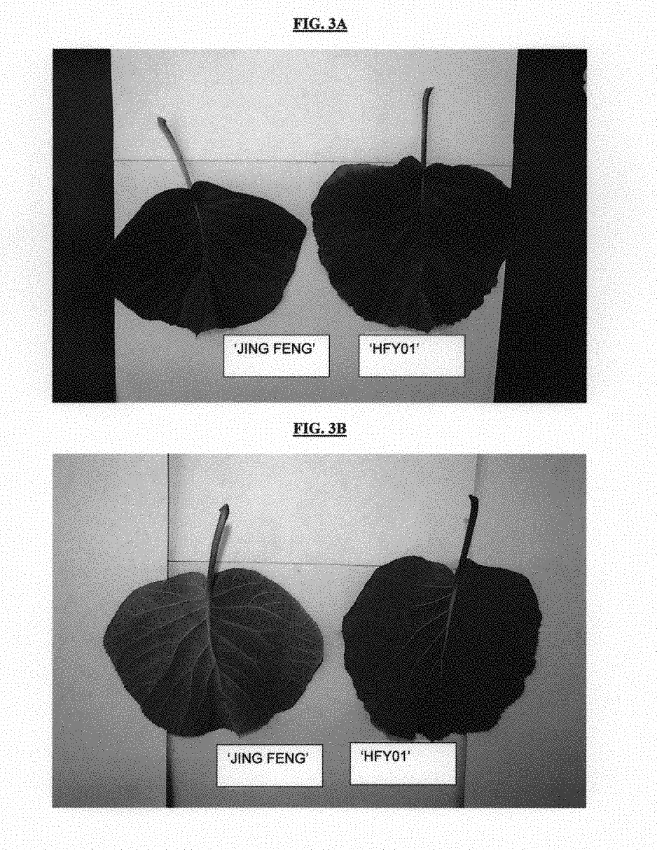

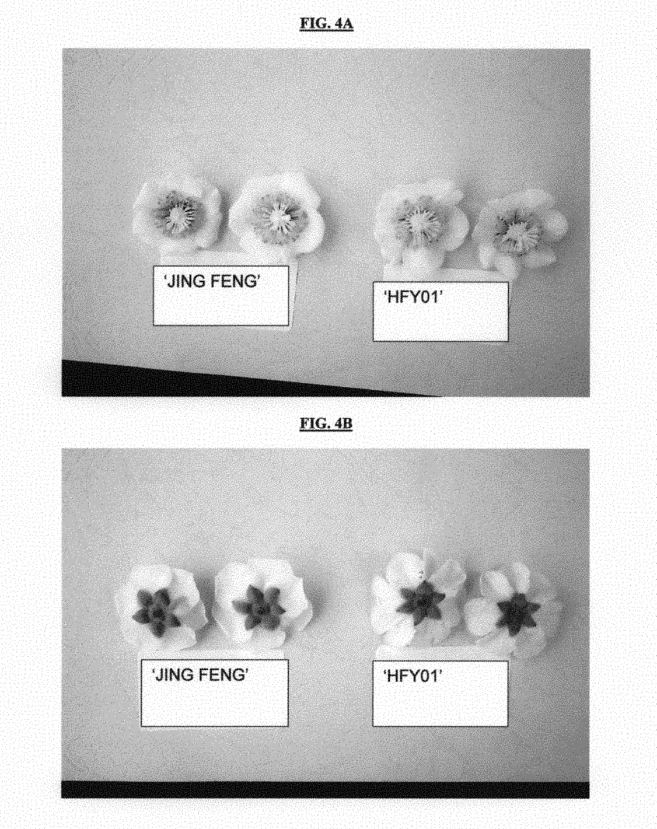

### DETAILED BOTANICAL DESCRIPTION

The new Actinidia cultivar ‘HFY01’ has not been observed under all possible environmental conditions. The phenotype of the new cultivar may vary with variations in environment such as temperature, light intensity, and day length without any change in the genotype of the Actinidia.

The aforementioned photographs, together with the following observations, measurements and values describe plants of ‘HFY01’ as grown outside in a field in Shifang county, Sichuan Province, China, under conditions which closely approximate those generally used in commercial practice.

Unless otherwise stated, the detailed botanical description includes observations, measurements and values based on 4 year old ‘HFY01’ plants grown in the field in Shifang county, Sichuan Province, China from 2009 to 2013. Quantified measurements are expressed as an average of measurements taken from a number of plants of ‘HFY01’. The measurements of any individual plant, or any group of plants, of the new cultivar may vary from the stated average.

Color references are made to The Royal Horticultural Society Colour Chart (R.H.S.), 4^{th} Edition, except where general colors of ordinary significance are used. Color values were taken under daylight conditions at approximately 10:00 AM in Shifang county, Sichuan Province, China.

All of the plants of ‘HFY01’, insofar as they have been observed, have been identical in all the characteristics described below.
Classification: Botanical: Actinidia chinensis pl. Commercial: ‘HFY01’Common: Kiwifruit, kiwi, Chinese gooseberry, Yang-tao.
Parentage:
   Female or seed parent: Actinidia cultivar designated ‘JXFX-CK04136’ (unpatented).
   Male or pollen parent.—Open Pollination.
Propagation: grafting on to rootstocks of Actinidia deliciosa C. F Liang.
Growing conditions: Light intensities: Full sunlight or slight shade. Temperature: During day, grown in range of 16° C. to 37° C., and during evening, grown in range of 10° C. to 28° C. Avoid frost injury. Irrigation: Amble irrigation during growing season with good drainage. Fertilization: Plants prefer and thrive in soil rich in organic matter. Heavy nitrogen feeders during the first half of growing season. pH: 5.0 to 6.5. Growth regulators: no use. Pruning or trimming requirements: For best fruit production, “cane replacement” pruning during the winter season when plant is dormant. Pruned plants will remain more compact and bear larger fruit.
Plant:
   Sex expression.—Female.
   Age.—Observed plants were 4 years old.
   Vigor.—Vigorous growth.
   Habit.—Woody, twining vine (liana), supported by fence system.
   Size at maturity (area covered by one plant).—Length: Typical: About 3 m to 4 m Observed: About 4 m to 5 m Width: Typical: About 2 m to 3 m Observed: About 3 m to 4 m Height: Typical: About 2 m to 3 m Observed: About 2 m to 3 m.
   Stem.—Length: About 2 m to 5 m. Diameter: About 8 mm to 15 mm Surface texture: Upper surface: downy Lower surface: downy Pith: present; pith type is hollow Pubescence (upper/lower surface): downy and aparese RHS 163A Bark Color: Upper surface: RHS 199A Lower surface: RHS 199C.
   Young shoot.—Length: Varies due to shape of plant; maximum of 3 m to 6 m; minimum of 30 cm/m to 60 cm/m. Diameter: About 5 mm to 30 mm Surface texture: Upper surface: downy Lower surface: downy Pubescence: absent Color: RHS 199B Upper surface: downy Lower surface: downy Color: RHS 199C Anthocyanin coloration of the growing tip: RHS 124B.
   Mature laterals.—Length: Varies due to shape of plant; maximum of 3 m to 6 m; minimum of 0.5 cm/m to 3.0 cm/m. Diameter: About 5 mm to 30 mm Surface texture: Upper surface: smooth Lower surface: smooth Pubescence: absent Color RHS 199B Upper surface: smooth Lower surface: smooth Color: 199C Upper surface: RHS 199B Lower surface: RHS 199C.
   Lenticels.—Length: About 0.2 mm to _0.5 mm Width: About 0.2 mm to 0.5 mm Color: RHS 155D Density: About 1 to 2/cm².
   Buds.—Shape: ellipsoid Length: About 0.6 Width: About 0.4 Color: Apex, RHS 23C, and base, RHS 34A Texture: deep Pubescence: absent Color RHS 23C.
   Leaf scar.—shallow.
   Leaves (mature).—Arrangement and Aspect: touching each other Size: Length: About 110 mm Width: About 120 mm Overall Shape: ovate.
   Apex shape.—acuminate Base shape: touch each other Distance between basal leaf lobes: About 50 mm. Margin: leaf blade: shape of apex is rounded Color: Upper surface: RHS 134A Under surface: RHS 134C Pubescence: none Puckering/blistering: Upper surface: weak Lower surface: weak Glaucosity: Upper surface: absent Lower surface: absent. Venation: Type: feather shaped venation from central vein to the leaf edge Color: RHS 132B.
   Leaf axil.—Color: RHS 132B Anthocyanin coloration: RHS 132B.
   Petiole.—Length: About 90 mm Diameter: About 2 mm Texture: sparsely pubescent Pubescence: very sparse to absent Color: RHS 131B Anthocyanin coloration (upper surface): RHS 131C.
   Stipule.—none.
Inflorescence:
   Blooming time.—Full bloom on or about April 25 in Sichuan Province, China.
   Blooming period.—About 7 to 9 days.
   Fragrance.—Typical of kiwifruit flower.
   Type.—cyme.
   Number of flowers per inflorescence.—About 3.
   Inflorescence size (terminal or king).—Diameter: About 4 cm Depth: About 4.5 cm.
   Petals.—Arrangement: overlapping Mean number per flower: range of 5 to 6 Size: Length: About 35 mm Width: About 30 mm Length/width ratio: 1:1.2 Overall shape: ovate Apex shape: acute Base shape: flat Texture (upper surface): weak Texture (lower surface): weak Margin: (please describe) Color (upper surface): Apex, RHS 155C, and base, RHS 155C Color (lower surface): Apex, RHS 155D, and base, RHS 155D.
   Sepals.—Arrangement: overlapping Mean number per flower: range of 5 to 6 Size: Length: About 20 mm Width: About 15 mm Length/width ratio: 1:3 Overall shape: ovate Apex shape: acuminate Base shape: flat Texture (upper surface): weak Texture (lower surface): weak Color (upper surface): Apex, RHS 132B and base, REIS 132B Color (lower surface): Apex, RHS 132C and base, RHS 132C.
   Pedicel.—Length: About 40 mm Diameter: About 0.2 mm Texture: smooth Color: RHS 131C.
Reproductive organs:
   Androecium.—Stamen: Number: About 20 to 30 Length: About 1 mm Color: RHS 19A Anthers: Length: About 2 mm Color: RHS 20A Filaments: Length: About 3 mm Color: RHS 20A Pollen: Amount: scarce Color: RHS 20A Pollination Requirements: Almost all Actinidia species are dioecious, so female cultivars must be interplanted with male pollinizers to ensure fruit production on the female cultivar.
   Gynoecium.—Pistils: Quantity: 36 Length: About 1 mm Color: RHS 155D Styles: Length: About 2 mm Width: About 1.5 mm Attitude: erect Stigmas: Length: About 3 mm Width: About 2 mm Ovary: Shape: ovoid. Length: About 3 mm. Width: About 2 mm. Pubescence: none.
Fruit:
   Keeping quality.—The fruit can be stored in cold temperature conditions for up to 12 weeks/months without losing firmness and juiciness. It has a shelf life up to 12-16 weeks without losing firmness and juiciness.
   Maturity when described.—harvest time is about 5 December in Sichuan Province, China. Fruit is ripe for eating at harvest.
   Firmness.—about 0.8 kg/cm².
   Maturity period after full bloom.—About 160 days after full bloom on month and date, i.e. April 25^{th.}
   Type.—Berry.
   Average fruit weight and size.—Weight: About 85 g Height: About 55 mm Diameter (at widest point): About 49 mm Diameter (at narrowest point): About 47 mm.
   Fruit shape.—General shape: long — trapezium Styler end description: weakly depressed Shoulder shape of stalk end: rounded Cross-section shape at median: transverse elliptic.
   Skin.—Firmness (at picking time): 4 to 5 kg/cm² Texture: smooth; lenticels are inconspicuous Pubescence: none Color: At harvest: RHS 164B During ripening: RHS 164B At maturity for consumption: RHS 164B.
   Flesh (at maturity for consumption).—Texture: tender and juicy Aroma: good Sugar content (at picking time): 17 to 18 Brix Acidity/Starch (at picking time) 1 to 1.4 g/lt Outer pericarp: Color: RHS 17A Inner pericarp (locules): Number (per fruit): About 45 Shape: oblong Length: About 55 mm Width: About 40 mm Color: RHS 17B Core: Shape and symmetry: triangle Diameter (maximum): About 8 mm Diameter (minimum): About 5 mm Woody spike: absent Color: RHS 155A.
Seeds: Number per fruit: About 1500-2000 Shape: cylindrical Length: About 1 mm Width: About 0.5 mm Texture: striped, smooth Color at maturity (in flesh): RHS 183A Color at maturity (when dried): RHS 164A.
Cultivar use: Fresh market.
Disease/pest resistance: PSA resistance is stronger.
Disease/pest susceptibility: some susceptibility to Pseudaulacaspis pentagona (White Peach Scale); Adoxophyes orana Fisher (Summer fruit tortrix); Acleris fimbriana Thunberg (Yellow Tortrix Moth); and Anomala corpulenta Motsch. (Scarabaeidae).
Winter hardiness: Tolerant to temperatures down to 0° C. without observed damage to wood and buds of dormant Actinidia plants; but open flowers and young fruitlets are killed by exposure to −5° C., depending on the length of exposure.
Drought/heat tolerance: Tolerant to temperatures up to 35° C., growth is limited by drought periods without irrigation.
Events: Time of vegetative budbreak: mid-February in Sichuan Province, China Time of beginning of flowering: end-April in Sichuan Province, China Time of maturity for harvest: early to December-in Sichuan Province, China.

I claim:

1. A new and distinct Actinidia chinensis Pl. plant named ‘HFY01’, as illustrated and described herein.
